(19) United States
(12) Patent Application Publication
(10) Pub. No.: US 2003/0055070 A1
(43) Pub. Date: Mar. 20, 2003

(54) Phosphodiesterase type 5 (PDE5) inhibitors for the treatment of selective serotonin reuptake inhibitor (SSR) induced sexual dysfunction

(76) Inventors: Wilma Harrison, Harrison, NY (US); Richard L. Siegel, New York, NY (US)

Correspondence Address: Gregg C. Benson, Pfizer Inc., Patent Department, MS 4159, Eastern Point Road, Groton, CT, 06340, US

(21) Appl. No.: 10/079,991

(22) Filed: Feb. 19, 2002

Related U.S. Application Data
(63) Continuation of application No. 09/602,790, filed on Jun. 23, 2000, now abandoned.
(60) Provisional application No. 60/141,980, filed on Jul. 1, 1999.

(51) Int. Cl. A61K 31/519; A61K 31/522; A61K 31/517; A61K 31/52

(52) U.S. Cl. 514/262.1; 514/263.1; 514/265.1; 514/266.1; 514/263.37

(57) ABSTRACT

This invention is directed to the use phosphodiesterase type 5 (PDE5) inhibitors in the treatment of selective serotonin reuptake inhibitor (SSRI) induced sexual dysfunction. Specifically, this invention is directed to a method for treating an animal to cure; prevent or ameliorate SSRI induced sexual dysfunction which comprises administering to the animal an effective amount of the inhibitor. The animal may be a male or a female human. The invention also includes the use of such inhibitors in the manufacture of a medicament to prevent; cure or ameliorate SSRI induced sexual dysfunction. Moreover, the invention includes a kit comprising a SSRI, such as sertraline, fluoxetine, paroxetine, and a PDE5 inhibitor, such as sildenafil citrate, for the treatment or prevention of serotonergic associated disorders such as depression, obsessive compulsive disorder or panic disorder, while reducing or preventing sexual dysfunction.

This application is a continuation of application No. 09/602,790 filed on Jun. 23, 2000, which claims priority from provisional application U.S. serial No. 60/141,980, filed Jul. 1, 1999.

### BACKGROUND OF THE INVENTION

This invention relates to the use of phosphodiesterase type 5 (PDE5 ) inhibitors for the treatment of selective serotonin reuptake inhibitor (SSRI) induced sexual dysfunction. In one embodiment the PDE5 inhibitors of this invention are pyrazolopyrimidone compounds, such as pyrazolo[4,3-d]pyrimidin-7-ones, or more specifically sildenafil citrate, VIAGRA®, 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate.

In the general male population, erectile impotence or erectile dysfunction (ED) may be defined as an inability to obtain or sustain an erection adequate for intercourse. Its prevalence is claimed to be between 2 and 7% of the human male population, increasing with age, up to 50 years, and between 18 and 75% between 55 and 80 years of age. In the U.S.A. alone, for example, it has been estimated that there are up to 10 million impotent males, with the majority suffering from problems of organic rather than of psychogenic origin.

Sildenafil citrate has been approved by the Food and Drug Administration (FDA) for the treatment of ED. Prior to the introduction of sildenafil citrate, reports of well-controlled clinical trials for treatment of ED were few and the efficacy of orally administered drugs was low. Although many different drugs had been shown to induce penile erection, they are only effective after direct injection into the penis, e.g. intraurethrally or intracavernosally (i.c.). Medical treatment using the i.c injection of vasoactive substances have been claimed to give good results with phenoxybenzamine, phentolamine, papaverine and prostaglandin E1, either alone or in combination; however, pain, priapism and fibrosis of the penis are associated with the i.c. administration of some of these agents. Potassium channel openers (KCO) and vasoactive intestinal polypeptide (VIP) have also been shown to be active i.c., but cost and stability issues could limit development of the latter. An alternative to the i.c. route is the use of glyceryl trinitrate (GTN) patches applied to the scrotum or inner thigh or a paste applied to the penis, which has been shown to be effective but produces side-effects in both patient and partner.

As a general alternative to pharmacological intervention, a variety of penile prostheses have been used to assist achievement of an erection. The short term success rate is good, but problems with infection and ischemia, especially in diabetic men, make this type of treatment a final option rather than first-line therapy. In addition, vacuum constriction devices have also been used to assist in the achievement of an erection.

Normal penile erection depends on the relaxation of smooth muscles in the corpora cavernosa. In response to sexual stimuli, cavernous nerves and endothelial cells release nitric oxide, which stimulates the formation of cyclic guanosine monophosphate (GMP) by guanylate cyclase. Sildenafil citrate is a selective inhibitor of cyclic-GMP-specific PDE5, the predominant isozyme metabolizing cyclic GMP in the corpus cavernosum. By selectively inhibiting cyclic-GMP catabolism in cavernosal smooth-muscle cells, sildenafil citrate restores the natural erectile response to sexual stimulation but does not cause erections in the absence of such stimulation. See, Derry et al. (1998) “Efficacy and Safety of Oral Sildenafil (Viagra) in Men with Erectile Dysfunction Caused by Spinal Cord Injury,” Neurology 51:1629-1633, Dinsmore et al. (1999) “Sildenafil Citrate (VIAGRA) in Erectile Dysfunction: Near Normalization in Men With Broad-Spectrum Erectile Dysfunction Compared with Age-Matched Healthy Control Subjects,” Urology 53:800-805; Goldstein et al. (1998) “Oral Sildenafil in the Treatment of Erectile Dysfunction,” NEJM 338(20): 1397-1404; Padma-Nathan et al. (1998) “Efficacy and Safety of Oral Sildenafil in the Treatment of Erectile Dysfunction: A Double-Blind, Placebo-Controlled Study of 329 Patients,” Int. J. Clin. Pract. 52(6):375-380; or Rendell et al. (1999) “Sildenafil for the Treatment of Erectile Dysfunction in Men With Diabetes,” JAMA 281 (5):421-426; the contents of which are hereby incorporated by reference in their entirety into the present application.

Pyrazolopyrimidones such as sildenafil are potent inhibitors of cyclic guanosine 3′,5′-monophosphate phosphodiesterases (cGMP PDEs) but not of cyclic adenosine 3′,5′-monophosphate phosphodiesterases (cAMP PDEs). This selective enzyme inhibition leads to elevated cGMP levels which, in turn, provides the basis for the utilities already disclosed in U.S. Pat. No. 5,250,534, EP-A-0463756 and EP-A-0526004; the contents of which are hereby incorporated by reference in their entirety into the present application. These compounds are known to be useful for the treatment of stable, unstable and variant (Prinzmetal) angina, hypertension, pulmonary hypertension, congestive heart failure, atherosclerosis, conditions of reduced blood vessel potency e.g. post-percutaneous transluminal coronary angioplasty (post-PTCA), peripheral vascular disease, stroke, bronchitis, allergic asthma, chronic asthma, allergic rhinitis, glaucoma, and diseases characterized by disorders of gut motility, e.g., irritable bowel syndrome (IBS).

SSRIs are compounds that inhibit the central nervous system uptake of serotonin (5-hydroxytryptophan, 5-HT) while having reduced or limited affinity for other neurologically active receptors. See, “Clinical Practice Guidelines for the Treatment of Major Depression Vol. 2” Agency for Healthcare Policy and Research, U.S. Department of Health and Human Services #93-0551 (1993). Examples of SSRIs include, but are not limited to, citalopram, CELEXA®; fluoxetine hydrochloride, PROZAC®; fluvoxamine maleate, LUVOX®; paroxetine hydrochloride, PAXIL®; sertraline hydrochloride, ZOLOFT®; and venlafaxine hydrochloride, EFFEXOR® or EFFEXOR® ER.

A common side effect of treatment with the SSRI class of antidepressants is sexual dysfunction including anorgasmia, the inability to achieve orgasm, delayed orgasm, delayed ejaculation, inability to ejaculate, erectile dysfunction, decreased libido, insufficient vaginal lubrication and general sexual dissatisfaction. See, Montejo-Gonzales et al. (1997) “SSRI-Induced Sexual Dysfunction: Fluoxetine, Paroxetine, Sertraline, and Fluvoxamine in a Prospective, Multicenter, and Descriptive Clinical Study” J. Sex & Marital Therapy 23:176-194. Since SSRIs must generally be taken for prolonged periods, ranging from months to years, sexual dysfunction can be a cause of poor compliance with SSRI treatment. This can result in relapse or serious worsening of the illness for which the SSRI was prescribed. Serotonin is believed to play an important role in sexual dysfunctions by inhibiting libido, ejaculation and orgasm. Because SSRI affects the central nervous system, it is believed that in males taking SSRIs who experience ED, the ED results in most cases from persistent anorgasmia as a secondary sign of the CNS side effects of the SSRI.

PDE5 inhibitors such as sildenafil act peripherally to increase blood flow and penile erectile response. PDE5 inhibitors would not be expected to have an influence on the centrally acting SSRI induced side effects such as decreased libido, anorgasmia, delayed orgasm and delayed ejaculation. Therefore, most strategies have focused on reversing CNS effects of SSRI. See, Lane (1997) “A Critical Review of Selective Serotonin Reuptake Inhibitor-Related Sexual Dysfunction; Incidence, Possible Aetiology and Implications for Management,” J. Psycho Pharmacology 11 (1):72-82

Recently other workers have reported the use of sildenafil citrate for the treatment of SSRI induced sexual dysfunction in both men and women. Fava et al. report a small scale study including nine men, five women, twelve of which were taking SSRIs (fluoxetine N=3, sertraline N=6, paroxetine N=2, and fluvoxamine N=1). Two were taking mirtazapine an antidepressant that acts as an antagonist of alpha-2 receptors and serotonin 5-HT-2 receptors. All patients showed statistically significant improvement in sexual functioning. Fava et al. (1998) “An Open Trial of Oral Sildenafil in Antidepressant-Induced Sexual Dysfunction” Psychotherapy and Psychosomatics 67:328-331.

Nurnberg et al. recently reported case studies of four patients (two men, two women) who had experienced SSRI induced sexual dysfunction. On taking sildenafil all reported a rapid reversal of SSRI induced sexual dysfunction. Nurnberg et al. (1999) “Sildenafil for Iatrogenic Serotonergic Antidepressant Medication-Induced Sexual Dysfunction in 4 Patients” J. Clin. Psychiatry 60(1):33-35. In two letters to the editor, Schaller at al. and Rosenberg report additional case studies of patients taking SSRIs responding well to treatment with sildenafil. Schaller et al. and Rosenberg (1999) “Sildenafil Citrate for SSRI-Induced Sexual Side Effects” Am. J. Psychiatry 156(1):156-157. The contents of these papers are hereby incorporated in their entirety into the present application.

### SUMMARY OF THE INVENTION

It has now been found that the PDE5 inhibitors of this invention are useful in the treatment of SSRI induced sexual dysfunction. Specifically, this invention is directed to a method for treating an animal to cure or prevent SSRI induced sexual dysfunction which comprises administering to the animal an effective amount of the compound. The animal can be a male or a female human. The invention also includes the use of such compounds to prevent or cure SSRI induced sexual dysfunction. Moreover, the invention includes a kit comprising a SSRI, such as sertraline, fluoxetine, paroxetine, and a PDE5 inhibitor or a pharmaceutically acceptable salt thereof, such as sildenafil citrate, for the treatment or prevention of a serotonergic associated disorder such as depression, obsessive compulsive disorder or panic disorder, while reducing or preventing sexual dysfunction.

### BRIEF DESCRIPTION OF THE FIGURES

FIGS. 1A and 2B show baseline and endpoint scores, standard error of mean (SEM), SEM for questions assessing the ability to achieve (Question 3; Panel A(*p<0.0001 versus placebo, ** p<0.001 versus placebo)) and ability to maintain (Question 4; Panel B (*p<0.0001 versus placebo, ** p<0.01 versus placebo)) an erection sufficient for sexual intercourse. The vertical scale for FIGS. 2-4 is as follows: 5 is “Almost always/always,” 4 is “Most times,” 3 is “Sometimes,” 2 is “A few times,” 1 is “Almost Never/Never” and 0 is “No activity.” *p<0.0001 versus placebo, **p<0.001 versus placebo.

FIGS. 2A and 2B show baseline and endpoint scores + SEM for questions assessing the frequency of ejaculation (Question 9; Panel A (*p<0.0001 versus placebo, **p<0.02 versus placebo)) and frequency of orgasm (Question 10; Panel B (*p<0.0001 versus placebo, ** p<0.01 versus placebo)).

FIGS. 3A and 3B show baseline and endpoint scores + SEM for questions assessing desire frequency (Question 11; Panel A (*p<0.001 versus placebo, ** p<0.02 versus placebo)) and desire level (Question 12; Panel B (* p<0.0001 versus placebo, ** p<0.01 versus placebo)).

### DETAILED DESCRIPTION

The present invention is directed to a method for the curative or prophylactic treatment of selective serotonin reuptake inhibitor (SSRI) induced sexual dysfunction in an animal, including a human which comprises administering to the animal an effective amount of a phosphodiesterase type 5 (PDE5) inhibitor. For example, the compound may be (i) a 5-substituted pyrazolo [4,3-d]pyrimidine-7-one as disclosed in European patent application 0201188; (ii) a griseolic acid derivative as disclosed in European patent applications Nos. 0214708 and 0319050; (iii) a 2-phenylpurinone derivative as disclosed in European patent application 0293063; (iv) a phenylpyridone derivative as disclosed in European patent application 0347027; (v) a fused pyrimidine derivative as disclosed in European patent application 0347146; (vi) a condensed pyrimidine derivative as disclosed in European patent application 0349239; (vii) a pyrimidopyrimidine derivative as disclosed in European patent application 0351058; (viii) a purine compound as disclosed in European patent application 0352960; (ix) a quinazolinone derivative as disclosed in European patent application 0371731; (x) a phenylpyrimidone derivative as disclosed in European patent application 0395328; (xi) an imidazoquinoxalinone derivative or its aza analogue as disclosed in European patent application 0400583; (xii) a phenylpyrimidone derivative as disclosed in European patent application 0400799; (xiii) a phenylpyridone derivative as disclosed in European patent application 0428268; (xiv) a pyrimidopyrimidine derivative as disclosed in European patent 0442204; (xv) a 4-aminoquinazoline derivative as disclosed in European patent application 0579496; (xvi) a 4,5-dihydro4-oxo-pyrrolo[1,2-a]quinoxaline derivative or its aza analogue as disclosed in European patent application 0584487; (xvii) a polycyclic guanine derivative as disclosed in International patent application WO 91/19717; (xviii) a nitrogenous heterocyclic compound as disclosed in International patent application WO 93/07124; (xix) a 2-benzyl-polycyclic guanine derivative as disclosed in International patent application WO 94/19351; (xx) a quinazoline derivative as disclosed in U.S. Pat. No. 4,060,615; (xxi) a 6-heterocyclyl pyrazolo [3,4-d]pyrimidin-4-one as disclosed in U.S. Pat. No. 5,294,612; (xxii) a benzimidazole as disclosed in Japanese patent application 5-222000; (xxiii) a cycloheptimidazole as disclosed in European Journal of Pharmacology, 251, (1994), 1; (xxiv) a N-containing heterocycle as disclosed in International patent application WO94/22855; (xxv) a pyrazolopyrimidine derivative as disclosed in European patent application 0636626; (xxvi) a 4-aminopyrimidine derivative as disclosed in European patent application 0640599; (xxvii) a imidazoquinazoline derivative as disclosed in International patent application WO 95/06648; (xxviii) an anthranilic acid derivative as disclosed in International patent application WO 95/18097; (xxix) a 4-aminoquinazoline derivative as disclosed in U.S. Pat. No. 5,436,233; (xxx) a tetracyclic derivative as disclosed in International patent application WO 95/19978; (xxxi) a imidazoquinazoline derivative as disclosed in European patent application 0668280; or (xxxii) a quinazoline compound as disclosed in European patent application 0669324; or (xxxiii) a tetracyclic compound as disclosed in International patent application WO 95/19978 or WO 97/03675, the contents of which are hereby incorporated by reference in their entirety into the present application.

Of particular interest for use in the present invention are compounds disclosed in EP 0579496, WO 93/07124, U.S. Pat. No. 5,294,612 and WO 94/22855 (xv, xviii, xxi and xxiv above); the compounds of EP 0579496 and WO 94/22855 being especially preferred. Additional examples of preferred compounds may be found in WO 96/16644 on pages 4-6, the contents of which are hereby incorporated by reference in their entirety into the present application.

In one embodiment, the present invention concerns the use of a compound of formula (I):

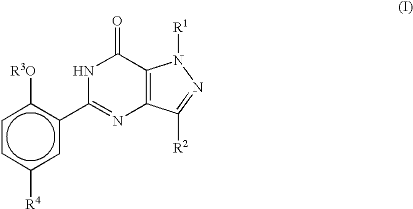

wherein R¹ is H; C₁-C₃ alkyl; C₁-C₃ perfluoroalkyl; or C₃-C₅ cycloalkyl;

R² is H; C₁-C₆ alkyl optionally substituted with C₃-C₆ cycloalkyl; C₁-C₃ perfluoroalkyl; or C₃-C₆ cycloalkyl;

R³ is C₁-C₆ alkyl optionally substituted with C₃-C₆ -cycloalkyl; C₁-C₆ perfluoroalkyl; C₃-C₅ cycloalkyl; C₃-C₆ alkenyl; or C₃-C₆ alkynyl;

R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;

R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;

R⁷ is H or C₁-C₄ alkyl;

R⁸ is C₁-C₃ alkyl optionally substituted with NR⁵R⁶;

R⁹ and R¹⁰ together with the nitrogen atom to which they are attached form a pyrrolidinyl, piperidino, morpholino or 4-N(R¹²) piperazinyl group wherein said group is optionally substituted with C₁-C₄ alkyl, C₁-C₃ alkoxy, NR¹³R¹⁴ or CONR¹³R¹⁴;

R¹¹ is H; C₁-C₃ alkyl optionally substituted with phenyl; (hydroxy)C₂-C₃ alkyl; or C₁-C₄ alkanoyl;

R¹² is H; C₁-C₆ alkyl; (C₁-C₃ alkoxy)C₂-C₆ alkyl; (hydroxy) C₂-C₆ alkyl; (R¹³R¹⁴N)C₂-C₆ alkyl; (R¹³R¹⁴NOC) C₁-C₆ alkyl; CONR¹³R¹⁴; CSNR¹³R¹⁴; or C(NH)NR¹³R¹⁴; and

R¹³ and R¹⁴ are each independently H; C₁-C₄ alkyl; (C₁-C₃ alkoxy)C₂-C₄ alkyl; or (hydroxy)C₂-C₄ alkyl; or a pharmaceutically acceptable salt thereof, or a pharmaceutical composition containing either entity.

In the above definition, unless otherwise indicated, alkyl groups having three or more carbon atoms, alkenyl and alkynyl groups having four or more carbon atoms, alkoxy groups having three carbon atoms and alkanoyl groups having four carbon atoms may be straight chain or branched chain. Halo means fluoro, chloro, bromo or iodo.

The compounds of formula (I) may contain one or more asymmetric centers and thus they can exist as enantiomers or diastereoisomers. Furthermore, certain compounds of formula (I) which contain alkenyl groups may exist as cis-isomers or trans-isomers. In each instance, the invention includes both mixtures and separate individual isomers.

The compounds of formula (I) may also exist in tautomeric forms and the invention includes both mixtures and separate individual tautomers.

The pharmaceutically acceptable salts of the compounds of formula (I) which contain a basic center are, for example, nontoxic acid addition salts formed with inorganic acids such as hydrochloric, hydrobromic, sulphuric and phosphoric acid, with organo-carboxylic acids, or with organo-sulphonic acids. Compounds of formula (I) can also provide pharmaceutically acceptable metal salts, in particular nontoxic alkali metal salts, with bases. Examples include the sodium and potassium salts.

A preferred group of compounds of formula (I) is that wherein R¹ is H, methyl or ethyl; R² is C₁-C₃ alkyl; R³ is C₂-C₃ alkyl or allyl; R⁴ is C₁-C₂ alkyl optionally substituted with OH, NR⁵R⁶, CN, CONR⁵R⁶ or CO₂R⁷; acetyl optionally substituted with NR⁵R⁶; hydroxyethyl optionally substituted with NR⁵R⁶; ethoxymethyl optionally substituted with OH or NR⁵R⁶; CH═CHCN; CH═CHCONR⁵R⁶; CH═CHCO₂R⁷; CONR⁵R⁶; CO₂H; Br; NR⁵R⁶; NHSO₂NR⁵R⁶; NHSO₂R⁸; SO₂NR⁹R¹⁰; or pyridyl or imidazolyl either of which is optionally substituted with methyl; R⁵ and R⁶ are each independently H, methyl or ethyl, or together with the nitrogen atom to which they are attached form a piperidino, morpholino, 4 N(R¹¹)-piperazinyl or imidazolyl group wherein said group is optionally substituted with methyl or OH; R⁷ is H or t-butyl; R⁸ is methyl or CH₂CH₂CH₂NR⁵R⁶; R⁹ and R¹⁰ together with the nitrogen atom to which they are attached form a piperidino or 4-N(R¹²)-piperazinyl group wherein said group is optionally substituted with NR¹³R¹⁴ or CONR¹³R¹⁴; R¹¹ is H, methyl, benzyl, 2 hydroxyethyl or acetyl; R¹² is H, C₁-C₃ alkyl, (hydroxy)C₂-C₃ alkyl, CSNR¹³R¹⁴ or C(NH)NR¹³R¹⁴; and R¹³ and R¹⁴ are each independently H or methyl.

A more preferred group of compounds of formula (I) is that wherein R¹ is methyl or ethyl; R² is C¹-C³ alkyl; R³ is ethyl, n-propyl or allyl; R⁴ is CH₂NR⁵R⁶, COCH₂NR⁵R⁶, CH(OH)CH₂NR⁵R⁶, CH₂OCH₂CH₃, CH₂OCH₂CH₂OH, CH₂OCH₂CH₂NR⁵R⁶, CH═CHCON (CH)₂CH═CHCO₂R⁷, CONR⁵R⁶, CO₂H, Br, NHSO₂NR5R⁶, NHSO₂CH₂CH₂CH₂NR⁵R⁶, SO₂NR⁹R¹⁰, 2-pyridyl, 1-imidazolyl or 1-methyl-2-imidazolyl; R⁵ and R⁶ together with the nitrogen atom to which they are attached form a piperidino, 4-hydroxypiperidino, morpholino, 4-N (R¹¹)-piperazinyl or 2-methyl-imidazolyl group; R⁷ is H or t-butyl; R⁹ and R¹⁰ together with the nitrogen atom to which they are attached form a 4-carbamoylpiperidino or 4-N(R¹²)piperazinyl group; R¹¹ is H, methyl, benzyl, 2-hydroxyethyl or acetyl; and R¹² is H, C₁-C₃ alkyl, 2-hydroxyethyl or CSNH₂.

A particularly preferred group of compounds of formula (I) is that wherein R¹ is methyl or ethyl; R² is n-propyl; R³ is ethyl, n-propyl or allyl; R⁴ is COCH₂NR⁵R⁶, CONR⁵R⁶, SO₂NR⁹R¹ or 1-methyl-2-imidazolyl; R⁵ and R⁶ together with the nitrogen atom to which they are attached form a morpholino or 4-N(R¹¹)-piperazinyl group; R⁹ and R¹⁰ together with the nitrogen atom to which they are attached form a 4-N(R¹²)-piperazinyl group; R¹¹ is methyl or acetyl; and R¹² is H, methyl, 2-propyl or 2-hydroxyethyl.

Especially preferred individual compounds of the invention include:

5-(2-ethoxy-5-morpholinoacetylphenyl)-1-methyl-3-n-propyl-1,6-dihydro-7H-pyrazolo [4,3-d]pyrimidin-7-one;

5-(5-morpholinoacetyl-2-n-propoxyphenyl)-1-methyl3-n-propyl-1,6-dihydro-7H-pyrazolo [4,3-d]pyrimidin-7-one;

1-[[3-(6,7-dihydro-1-methyl-7-oxo-3-propyl-1H-pyrazolo[4,3-d]pyrimidin-5-yl )-4-ethoxyphenyl]sulfonyl]-4-methylpiperazine;

5-[2-allyloxy-5-(4-methyl-1-piperazinylsulphonyl)-phenyl]-1-methyl-3-n-propyl-1,6-dihydro-7H- pyrazolo[4,3-d]pyrimidin-7-one;

5-[2-ethoxy-5-[4-(2-propyl)-1-piperazinyl-sulphonyl]phenyl]- 1-methyl-3-n-propyl-1,6-dihydro-7H-pyrazolo[4,3-d]pyrimidin-7-one;

5-[2-ethoxy-5-[4-(2-hydroxyethyl)-1-piperazinsulphonyl]phenyl]-1-methyl-3-n-propyl-1,6-dihydro-7H-pyrazolo[4,3-d]pyrimidin-7-one;

5-[5-[4-(2-hydroxyethyl)-1-piperazinylsulphonyl]-2-n-propoxyphenyl]-1-methyl-3-n-propyl-1,6-dihydro-7H-pyrazolo[4,3-d]pyrimidin-7-one;

5-[2-ethoxy-5-(4-methyl-1-piperazinylcarbonyl)-phenyl]-1-methyl-3-n-propyl-1,6-dihydro-7H-pyrazolo[4,3-d]pyrimidin-7-one; and

5-[2-ethoxy-5-(1-methyl-2-imidazolyl)phenyl] methyl-3-n-propyl-1,6-dihydro-7H-pyrazolo [4,3-d]pyrimidin-7-one.

The PDE5 inhibitors of this invention are useful for the manufacture of a medicament for the curative or prophylactic treatment of SSRI sexual dysfunction in an animal, including a human. The human may be male or female.

The PDE5 inhibitors and their pharmaceutically acceptable salts, processes for the preparation thereof, in vitro test methods for determining the cGMP PDE and cAMP PDE inhibitory activities thereof, pharmaceutical compositions thereof, and routes of administration for human use, are described in U.S. Pat. No. 5,250,534, WO94/28902, EP-A-0463756 and EP-A-0526004, the contents of which are hereby incorporated by reference in their entirety into the present application.

The PDE5 inhibitors of the invention are envisaged for the prophylactic or curative treatment of SSRI induced male sexual dysfunction and female sexual dysfunction. The sexual dysfunctions include but are not limited to anorgasmia, decreased libido, delayed ejaculation, delayed orgasm, dyspareunia (pain during intercourse), erectile dysfunction, general sexual dissatisfaction, inability to ejaculate or insufficient vaginal lubrication. Moreover, the PDE5 inhibitors of this invention may improve general sexual satisfaction for those taking SSRIs.

The SSRI may be taken for prevention or treatment of a variety of serotonergic associated disorders including, but not limited to, attention deficit disorder (ADD), bipolar disorder, bulemia, depression, dysthymic disorder, generalized anxiety disorder, impulse control disorders, major depressive disorder, obsessive compulsive disorder (OCD), panic disorder, post-traumatic stress disorder (PTSD), premenstrual dysphoric disorder (PMDD), premenstrual syndrome (PMS), or social phobia.

SSRIs are compounds that selectively inhibit the central nervous system uptake of serotonin (5-hydroxytryptophan, 5-HT) while having reduced or limited affinity for other neurologically active receptors. Examples of SSRIs include, but are not limited to, citalopram, CELEXA®; fluoxetine hydrochloride, PROZAC®; fluvoxamine maleate, LUVOX®; paroxetine hydrochloride, PAXIL®; sertraline hydrochloride, ZOLOF®; and venlafaxine hydrochloride, EFFEXOR® or EFFEXOR® ER.

Moreover, the invention also includes methods, uses and kits comprising a PDE5 inhibitor and other antidepressants associated with sexual dysfunction including tricyclic antidepressants (TCAs), such as clomipramine, desipramine, imipramine, nortriptyline; monoamine oxidase inhibitors (MAOIs); or other compounds such as trazodone, mirtazapine, or nefazadone.

Generally, in humans, oral administration of the PDE5 inhibitors of the invention is the preferred route, being the most convenient and avoiding the disadvantages associated with i.c. administration. A preferred dosing regimen for a typical person is 5 to 200 mg of a compound of formula (I) one hour before sexual activity. The preferred dosing regimen is 25 to 100 mg of a compound one hour before sexual activity. Moreover, the invention provides a method of prevention or treatment of persistent SSRI induced sexual dysfunction. The prevention or treatment of the persistent sexual dysfunction may involve taking the PDE5 inhibitor daily, several times a week, weekly, several times a month, or monthly. In circumstances where the recipient suffers from a swallowing disorder or from impairment of drug absorption after oral administration, the drug may be administered parenterally.

For veterinary use, PDE5 inhibitor or a nonionic salt thereof is administered as a suitably acceptable formulation in accordance with normal veterinary practice and the veterinary surgeon will determine the dosing regimen and route of administration which will be most appropriate for a particular animal.

Thus, the invention includes a pharmaceutical composition for the curative or prophylactic treatment of SSRI induced sexual dysfunction in an animal, including man, comprising a PDE5 inhibitor, or a pharmaceutically acceptable salt thereof, together with a pharmaceutically acceptable diluent or carrier.

The invention also provides a kit or a composition for the prevention or treatment of an SSRI related disorder comprising a PDE5 inhibitor as described above and an SSRI. In one embodiment, the SSRI may be citalopram, sertraline, fluoxetine, paroxetine or fluvoxamine or a pharmaceutically acceptable salt thereof.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of PDE5 inhibitor, a prodrug thereof or a salt of such compound or prodrug and a second compound, an SSRI as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, where appropriate for a PDE5 inhibitor, a daily dose of can consist of one tablet or capsule while a daily dose of the SSRI can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The following example is presented for purposes of illustration only and are not intended to limit the scope of the invention in any way.

### EXAMPLE

Summary/Overview

A retrospective analysis of combined data from 10 Phase II/III double-blind, placebo-controlled, fixed- and flexible-dose trials identified a subgroup of men with ED who were receiving 5-200 mg of sildenafil citrate (S) or placebo (P) and taking concomitant SSRIs. Studies of sildenafil citrate in various populations have been reported. For example, Goldstein et al. report the results of two sildenafil citrate studies in men. In one study 532 men were treated with oral sildenafil citrate (25, 50 or 100 mg) or placebo over 24 weeks to study dose-response. In the second study 329 men were treated with sildenafil citrate or placebo with dose escalation to 100 mg based on efficacy and tolerance. Additional studies may be found in the following references: Derry et al. (1998), Dinsmore et al. (1999), Goldstein et al. (1998), Padma-Nathan et al. (1998) and Rendell et al. (1999). Analysis of efficacy (ANCOVA) included responses to several questions including question 9 (Q9; frequency of ejaculation) and question 10 (Q10; frequency of orgasm) of the International Index of Erectile Function. In the International Index of Erectile Function, sexual activity includes intercourse, caressing, foreplay and masturbation; sexual intercourse is defined as vaginal penetration of a partner by the respondent; sexual stimulation includes situations like foreplay with a partner, looking at erotic pictures, etc.; ejaculation: the ejection of semen from the penis (or the feeling of this). Question 9 is, “Over the past 4 weeks, when you had sexual stimulation or intercourse how often did you ejaculate?” Question 10 is, “Over the past 4 weeks, when you had sexual stimulation or intercourse how often did you have the feeling of orgasm (with or without ejaculation)? Scores are based on a scale from 1 (almost never or never) to 5 (almost always or always), with 0 representing “No sexual stimulation/intercourse.” Each question was scored from 0 to 5, with lower scores indicating greater sexual dysfunction. Results are expressed as mean baseline score's standard error of mean, (±SEM) and mean change from baseline (±SEM) at the end of treatment (6 to 24 weeks).

Results

| | | Sildenafil | Sildenafil | | | | |
|---|---|---|---|---|---|---|---|
| | | citrate | citrate | | Placebo | Placebo | P |
| Subgroup | N | (baseline) | (change) | N | (baseline) | (change) | value |
| Q9: With SSRIs | 62 | 2.42 ± 0.23 | 1.16 ± 0.25 | 30 | 2.67 ± 0.37 | 0.13 ± 0.29 | <0.05 |
| Without SSRIs | 2041 | 2.74 ± 0.04 | 0.90 ± 0.04 | 1112 | 2.69 ± 0.06 | 0.05 ± 0.05 | <0.001 |
| Q10: With SSRIs | 62 | 2.24 ± 0.22 | 1.34 ± 0.27 | 30 | 2.53 ± 0.34 | 0.23 ± 0.37 | <0.01 |
| Without SSRIs | 2050 | 2.65 ± 0.04 | 0.93 ± 0.04 | 1112 | 2.61 ± 0.05 | 0.11 ± 0.05 | <0.001 |

Sildenafil citrate significantly improved the frequency of ejaculations and orgasm in patients with ED taking concomitant SSRIs compared with subjects treated with placebo.

Methods

Study Design

This was a retrospective subanalysis of combined data from 10 double-blind, placebo-controlled, fixed- and flexible-dose studies with sildenafil citrate for the treatment of ED. The subanalysis was conducted to assess whether treatment with sildenafil citrate can alleviate symptoms of sexual dysfunction in men taking SSRIs. The studies lasted for 6 to 24 weeks and dosing of the study drug ranged from 5 mg to 200 mg of sildenafil citrate or matching placebo. Patients were instructed to take medication approximately 1 hour before anticipated sexual activity but not more than once daily.

Patients

A total of 3414 patients were randomized to treatment. The main inclusion criteria were as follows: Men aged 18 years and older; Clinical diagnosis of ED of more than 6 months' duration; Stable relationship with a female partner for at least 6 months prior to the study; and written informed consent. The main exclusion criteria were as follows: Penile anatomical deformities; Major illness or uncontrolled psychiatric disorder; Concomitant treatment with nitrates or nitric oxide donors, estrogens, androgens, or anticoagulants. Other treatments for sexual dysfunction were not allowed during the study.

Efficacy Assessments

Efficacy was assessed at the end of treatment (6 to 24 weeks) by responses to the validated 15-item International Index of Erectile Function (IIEF) questionnaire. Rosen et al. (1997) “The International Index of Erectile Function (IIEF): a Multidimensional Scale for Assessment of Erectile Dysfunction,” Urology 49:822-830. Responses were graded on a scale of 1 (“almost never/never”) to 5 (“almost always/always”), with a score of 0 indicating no sexual activity. Efficacy results were evaluated using analysis of covariance (ANCOVA) and were expressed as mean scores.

Results

Demographics

3414 patients entered the studies and 3036 (89%) patients completed treatment. Data for efficacy analysis were available for 3255 patients. Of these, 2112 patients received treatment with sildenafil citrate and 1143 patients received treatment with placebo. Ninety-three patients also received treatment with an SSRI: 62 patients in the sildenafil citrate treatment group and 31 patients in the placebo treatment group. Patient demographics are summarized in Table 1.

TABLE 1
Demographic Data

| | | Placebo + | Sildenafil | Sildenafil citrate + |
|---|---|---|---|---|
| | Placebo | SSRI | citrate | SSRI |
| | (N = 1112) | (N = 31) | (N = 2050) | (N = 62) |
| Mean age, yr (range) | 54.1 (19-89) | 51.2 (26-70) | 55.9 (19-87) | 53.8 (25-74) |
| Mean duration of ED, | 5.6 (0.4-38.4) | 4.9 (0.5-21.5) | 5.1 (0.1-40) | 5.7 (0.5-21.5) |
| yr (range) | | | | |
| Etiology of ED, number | | | | |
| (%) of patients | | | | |
| Organic | 65% | 52% | 60% | 68% |
| Psychogenic | 14% | 18% | 14% | 14% |
| Organic/psychogenic | 21% | 30% | 26% | 19% |

Efficacy Assessments of Sexual Function

The mean scores for questions assessing the abilities to achieve and maintain an erection sufficient for sexual intercourse increased from mean baseline scores by 80% and 107%, respectively, for patients receiving sildenafil citrate, and by 130% and 195%, respectively, for patients receiving sildenafil citrate and an SSRI (FIG. 1). The Improvements were significantly greater for patients in the sildenafil citrate treatment groups when compared with patients in the placebo treatment groups (overall p values<0.01).

Likewise similar improvements were seen for other aspects of erectile function (Table 2). Questions assessing erection frequency, erection firmness, maintenance ability, and erection confidence improved by 60%, 78%, 117%, and 76%, respectively, for patients receiving sildenafil citrate, and by 74%, 121%, 224%, and 99%, respectively, for patients receiving sildenafil citrate and an SSRI (overall p values<0.01 for comparison with placebo).

TABLE 2
Baseline and endpoint mean scores for questions assessing erectile function

| | | Treatment | | | | | | |
|---|---|---|---|---|---|---|---|---|
| IIEF Question | | N | Baseline | Endpoint | N | Baseline | Endpoint | P value |
| | | Placebo | | | Sildenafil citrate | | | |
| 1 | Erection | 1112 | 2.27 ± 0.05 | 2.26 ± 0.05 | 2067 | 2.27 ± 0.04 | 3.63 ± 0.04 | <0.0001 |
| | frequency | | | | | | | |
| 2: | Erection | 1112 | 1.96 ± 0.04 | 2.07 ± 0.05 | 2064 | 1.97 ± 0.03 | 3.50 ± 0.04 | <0.0001 |
| | firmness | | | | | | | |
| 5: | Maintenance | 1106 | 1.47 ± 0.04 | 1.77 ± 0.05 | 2055 | 1.54 ± 0.03 | 3.35 ± 0.04 | <0.0001 |
| | ability | | | | | | | |
| 15: | Erection | 1110 | 1.72 ± 0.03 | 1.95 ± 0.03 | 2047 | 1.76 ± 0.02 | 3.10 ± 0.03 | <0.0001 |
| | confidence | | | | | | | |
| | | Placebo + SSRI | | | Sildenafil citrate + SSRI | | | |
| 1: | Erection | 31 | 2.58 ± 0.32 | 2.77 ± 0.34 | 61 | 1.88 ± 0.21 | 3.28 ± 0.22 | 0.007 |
| | frequency | | | | | | | |
| 2: | Erection | 31 | 2.19 ± 0.28 | 2.16 ± 0.29 | 60 | 1.48 ± 0.16 | 3.27 ± 0.23 | <0.0001 |
| | firmness | | | | | | | |
| 5: | Maintenance | 31 | 1.55 ± 0.25 | 1.81 ± 0.26 | 62 | 1.03 ± 0.15 | 3.34 ± 0.23 | <0.0001 |
| | ability | | | | | | | |
| 15: | Erection | 31 | 1.42 ± 0.11 | 1.74 ± 0.18 | 61 | 1.51 ± 0.10 | 3.00 ± 0.18 | <0.0001 |
| | confidence | | | | | | | |

P values are calculated for comparison with placebo

The mean scores for questions assessing the frequencies of ejaculation and orgasm increased significantly in patients receiving sildenafil citrate (p values<0.0001 versus placebo) and in patients receiving sildenafil citrate and an SSRI (p<0.02 and p<0.01, respectively, versus placebo)(FIG. 2).

Moreover, questions assessing the quality of intercourse, such as intercourse frequency, intercourse satisfaction, and intercourse enjoyment, significantly improved for patients receiving sildenafil citrate with or without an SSRI (Table 3). The mean scores increased by 57%, 93%, and 70%, respectively, for patients receiving sildenafil citrate (p values<0.0001), and by 89%, 182%, and 128%, respectively, for patients receiving sildenafil citrate and an SSRI (p values<0.02).

TABLE 3
Baseline and endpoint mean scores for questions assessing intercourse satisfaction

| | | Treatment | | | | | | |
|---|---|---|---|---|---|---|---|---|
| IIEF Question | | N | Baseline | Endpoint | N | Baseline | Endpoint | P value |
| | | Placebo | | | Sildenafil citrate | | | |
| 6: | Intercourse | 1117 | 1.91 ± 0.04 | 2.55 ± 0.05 | 2061 | 2.00 ± 0.03 | 3.13 ± 0.03 | <0.0001 |
| | frequency | | | | | | | |
| 7: | Intercourse | 1118 | 1.64 ± 0.04 | 1.92 ± 0.05 | 2052 | 1.75 ± 0.03 | 3.38 ± 0.04 | <0.0001 |
| | Satisfaction | | | | | | | |
| 8: | Intercourse | 1115 | 1.79 ± 0.04 | 2.01 ± 0.05 | 2055 | 1.86 ± 0.03 | 3.16 ± 0.03 | <0.0001 |
| | enjoyment | | | | | | | |
| | | Placebo + SSRI | | | Sildenafil citrate + SSRI | | | |
| 6: | Intercourse | 31 | 1.61 ± 0.24 | 2.32 ± 0.28 | 61 | 1.59 + 0.17 | 3.00 + 0.21 | 0.0115 |
| | frequency | | | | | | | |
| 7: | Intercourse | 31 | 1.39 ± 0.25 | 1.97 ± 0.27 | 61 | 1.19 + 0.16 | 3.36 + 0.23 | 0.0008 |
| | satisfaction | | | | | | | |
| 8: | Intercourse | 31 | 1.84 ± 0.27 | 2.06 ± 0.28 | 61 | 1.36 + 0.18 | 3.10 + 0.20 | <0.0001 |
| | enjoyment | | | | | | | |

P values are calculated for comparison with placebo

The final scores for level of sexual desire showed a trend towards significance for sildenafil citrate and SSRI compared with placebo compared with SSRI (P=0.0581), whereas the analysis of the frequency of sexual desire was not statistically significant for patients receiving sildenafil citrate and an SSRI, when compared with placebo treatment (p=0.648)(FIG. 3).

However, overall satisfaction with sex life and satisfaction with the relationship also increased significantly for patients receiving sildenafil citrate treatment. The mean scores increased by 68% and 40%, respectively, for patients receiving sildenafil citrate (p values<0.0001 versus placebo), and by 90% and 66%, respectively, for patients receiving sildenafil citrate and an SSRI (p values<0.0001 versus placebo)(Table 4).

TABLE 4
Baseline and endpoint mean scores for questions assessing
overall satisfaction and relationship satisfaction

| | | Treatment | | | | | | |
|---|---|---|---|---|---|---|---|---|
| IIEF Question | | N | Baseline | Endpoint | N | Baseline | Endpoint | P value |
| | | Placebo | | | Sildenafil citrate | | | |
| 13: | Overall | 1115 | 1.95 ± 0.03 | 2.20 ± 0.04 | 2056 | 2.00 ± 0.02 | 3.36 ± 0.03 | <0.0001 |
| | satisfaction | | | | | | | |
| 14: | Relationship | 1107 | 2.59 ± 0.04 | 2.80 ± 0.04 | 2043 | 2.63 ± 0.03 | 3.68 ± 0.03 | <0.0001 |
| | satisfaction | | | | | | | |
| | | Placebo + SSRI | | | Sildenafil citrate + SSRI | | | |
| 13: | Overall | 31 | 1.74 ± 0.17 | 1.84 ± 0.19 | 61 | 1.66 ± 0.12 | 3.16 ± 0.18 | <0.0001 |
| | satisfaction | | | | | | | |
| 14: | Relationship | 31 | 2.82 ± 0.21 | 2.42 ± 0.22 | 61 | 2.18 ± 0.16 | 3.61 ± 0.18 | <0.0001 |
| | satisfaction | | | | | | | |

P values are calculated for comparison with placebo

Conclusions

In conclusion, treatment with sildenafil citrate significantly improved important aspects of sexual function in patients with ED with or without concomitant treatment with an SSRI. In addition, sildenafil citrate significantly improved the frequency of ejaculation and orgasm in men with ED receiving concomitant SSRI therapy.

The invention described and claimed herein is not to be limited in scope by the specific embodiments herein disclosed since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

We claim:

1. A method for the curative or prophylactic treatment of selective serotonin reuptake inhibitor (SSRI) induced sexual dysfunction in an animal, including a human which comprises administering to the animal an effective amount of a phosphodiesterase type 5 (PDE5) inhibitor.

2. The method of claim 1 wherein the phosphodiesterase inhibitor is a 5-substituted pyrazolo [4,3-d]pyrimidine-7-one, a griseolic acid derivative, a 2-phenylpurinone derivative, a phenylpyridone derivative, a fused pyrimidine, a condensed pyrimidine derivative, a pyrimidopyrimidine derivative, a purine compound, a quinazolinone derivative, a phenylpyrimidone derivative, an imidazoquinoxalinone derivative or its aza analogue, a phenylpyrimidone derivative, a phenylpyridone derivative, a pyrimidopyrimidine derivative, a 4-aminoquinazoline derivative, a 4,5-dihydro-4-oxo-pyrrolo[1,2-a]quinoxaline derivative or its aza analogue, a polycyclic guanine derivative, a nitrogenous heterocyclic compound, a 2-benzyl-polycyclic guanine derivative, a quinazoline derivative, a 6-heterocyclyl pyrazolo [3,4-d]pyrimidin-4-one, a benzimidazole, a cycloheptimidazole, or an N-containing heterocycle.

3. The method of claim 2 wherein the phosphodiesterase inhibitor is a 5-substituted pyrazolo [4,3-d]pyrimidine-7-one or a 6-heterocyclyl pyrazolo [3,4-d]pyrimidin-4-one.

4. The method of claim 3 wherein the phosphodiesterase inhibitor is a compound of formula (I): 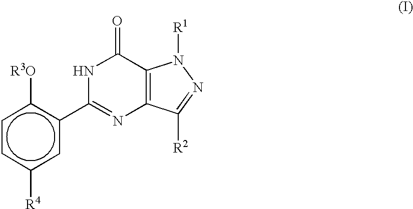
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆-cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²) piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy) C₂-C₆ alkyl; (R¹³R¹⁴N)C₂-C₆ alkyl; (R¹³R¹⁴NOC) C₁-C₆ alkyl; CONR¹³R¹⁴; CSNR¹³R¹⁴; or C(NH)NR¹³R¹⁴; and
R¹³ and R¹⁴ are each independently H; C₁-C₄ alkyl; (C₁-C₃ alkoxy)C₂-C₄ alkyl; or (hydroxy)C₂-C₄ alkyl; or a pharmaceutically acceptable salt thereof, or a pharmaceutical composition containing either entity.

5. The method of claim 4 wherein in the compound of formula (I) R¹ is H, methyl or ethyl; R² is C₁-C₃ alkyl; R³ is C₂-C₃ alkyl or allyl; R⁴ is C₁-C₂ alkyl optionally substituted with OH, NR⁵R⁶, CN, CONR⁵R⁶ or CO₂R⁷; acetyl optionally substituted with NR⁵R⁶; hydroxyethyl optionally substituted with NR⁵R⁶; ethoxymethyl optionally substituted with OH or NR⁵R⁶; CH═CHCN; CH═CHCONR⁵R⁶; CH═CHCO₂R⁷; CONR⁵R⁶; CO₂H; Br; NR⁵R⁶; NHSO₂NR⁵R⁶; NHSO₂R⁸; SO₂NR⁹R¹⁰; or pyridyl or imidazolyl either of which is optionally substituted with methyl; R⁵ and R⁶ are each independently H, methyl or ethyl, or together with the nitrogen atom to which they are attached form a piperidino, morpholino, 4 N(R¹¹)-piperazinyl or imidazolyl group wherein said group is optionally substituted with methyl or OH; R⁷ is H or t-butyl; R⁸ is methyl or CH₂CH₂CH₂NR⁵R⁶; R⁹ and R¹⁰ together with the nitrogen atom to which they are attached form a piperidino or 4-N(R¹²)-piperazinyl group wherein said group is optionally substituted with NR¹³R¹⁴ or CONR¹³R¹⁴; R¹¹ is H, methyl, benzyl, 2 hydroxyethyl or acetyl; R¹² is H, C₁-C₃ alkyl, (hydroxy)C₂-C₃ alkyl, CSNR¹³R¹⁴ or C(NH)NR¹³R¹⁴; and R¹³ and R¹⁴ are each independently H or methyl.

6. The method of claim 5 wherein in the compound of formula (I) R¹ is methyl or ethyl; R² is C¹-C³ alkyl; R³ is ethyl, n-propyl or allyl; R⁴ is CH₂NR⁵R⁶, COCH₂NR⁵R⁶, CH(OH)CH₂NR⁵R⁶, CH₂OCH₂CH₃, CH₂OCH₂CH₂OH, CH₂OCH₂CH₂NR⁵R⁶, CH═CHCON (CH)₂CH═CHCO₂R⁷, CONR⁵R⁶, CO₂H, Br, NHSO₂NR5R⁶, NHSO₂CH₂CH₂CH₂NR⁵R⁶, SO₂NR⁹R¹⁰, 2-pyridyl, 1-imidazolyl or 1-methyl-2-imidazolyl; R⁵ and R⁶ together with the nitrogen atom to which they are attached form a piperidino, 4-hydroxypiperidino, morpholino, 4-N (R¹¹)-piperazinyl or 2-methyl-limidazolyl group; R⁷ is H or t-butyl; R⁹ and R¹⁰ together with the nitrogen atom to which they are attached form a 4-carbamoylpiperidino or 4-N(R¹²)piperazinyl group; R¹¹ is H, methyl, benzyl, 2-hydroxyethyl or acetyl; and R¹² is H, C₁-C₃ alkyl, 2-hydroxyethyl or CSNH₂.

7. The method of claim 6 wherein in the compound of formula (I) R¹ is methyl or ethyl; R² is n-propyl; R³ is ethyl, n-propyl or allyl; R⁴ is COCH₂NR⁵R⁶, CONR⁵R⁶, SO₂NR⁹R¹⁰ or 1-methyl-2-imidazolyl; R⁵ and R⁶ together with the nitrogen atom to which they are attached form a morpholino or 4-N(R¹¹)-piperazinyl group; R⁹ and R¹⁰ together with the nitrogen atom to which they are attached form a 4-N(R¹²)-piperazinyl group; R¹¹ is methyl or acetyl; and R¹² is H, methyl, 2-propyl or 2-hydroxyethyl.

8. The method of claim 7 wherein the compound of formula (I) is selected from:
5-(2-ethoxy-5-morpholinoacetylphenyl )-1-methyl-3-n-propyl-1,6-dihydro-7H-pyrazolo [4,3-d]pyrimidin-7-one;
5(5-morpholinoacetyl-2-n-propoxyphenyl)-1-methyl3-n-propyl-1,6-dihydro-7H-pyrazolo [4,3-d]pyrimidin-7-one;
1-[[3-(6,7-dihydro-1-methyl-7-oxo-3-propyl-1H-pyrazolo[4,3-d]pyrimidin-5-yl )-4-ethoxyphenyl]sulfonyl]-4-methylpiperazine;
5-[2-allyloxy-5-(4-methyl-1-piperazinylsulphonyl)-phenyl]-1-methyl-3-n-propyl-1,6-dihydro-7H- pyrazolo[4,3-d]pyrimidin-7-one;
5-[2-ethoxy-5-[4-(2-propyl)-1-piperazinyl-sulphonyl]phenyl]-1-methyl-3-n-propyl-1,6-dihydro-7H-pyrazolo [4,3-d]pyrimidin-7-one;
5-[2-ethoxy-5-[4-(2-hydroxyethyl)-1-piperazinsulphonyl]phenyl]-1-methyl-3-n-propyl-1,6-dihydro-7H-pyrazolo[4,3-d]pyrimidin-7-one;
5-[5-[4-(2-hydroxyethyl)-1-piperazinylsulphonyl]-2-n-propoxyphenyl]-1-methyl-3-n-propyl-1,6-dihydro-7H-pyrazolo [4,3-d]pyrimidin-7-one;
5-[2-ethoxy-5-(4-methyl-1-piperazinylcarbonyl)-phenyl]-1-methyl-3-n-propyl-1,6-dihydro-7H-pyrazolo[4,3-d]pyrimidin-7-one; and
5-[2-ethoxy-5-(1-methyl-2-imidazolyl)phenyl] methyl-3-n-propyl-1,6-dihydro-7H-pyrazolo [4,3-d]pyrimidin-7-one.

9. The method of claim 8 wherein the compound of formula (I) is 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine.

10. The method of claims 1 or 9 wherein the animal is a male animal.

11. The method of claim 10 wherein the male animal is a human.

12. The method of claims 1 or 9 wherein the animal is a female animal.

13. The method of claim 13 wherein the female animal is a human.

14. The method according to claims 1 or 9 wherein the sexual dysfunction is anorgasmia, decreased libido, delayed ejaculation, delayed orgasm, dyspareunia, erectile dysfunction, general sexual dissatisfaction, inability to ejaculate or insufficient vaginal lubrication.

15. The method of claim 14 wherein the sexual dysfunction is anorgasmia.

16. The method of claim 14 wherein the sexual dysfunction is delayed orgasm.

17. The method of claim 14 wherein the sexual dysfunction is decreased libido.

18. The method of claims 1 or 9 wherein the selective serotonin reuptake inhibitor (SSRI) is associated with curative or prophylactic treatment of a variety of serotonergic associated disorders including, but not limited to, attention deficit disorder (ADD), bipolar disorder, bulemia, depression, dysthymic disorder, generalized anxiety disorder, impulse control disorders, major depressive disorder, obsessive compulsive disorder (OCD), panic disorder, post-traumatic stress disorder (PTSD), premenstrual dysphoric disorder (PMDD), premenstrual syndrome (PMS), or social phobia.

19. The method of claims 1 or 9 wherein the selective serotonin reuptake inhibitor (SSRI) is citalopram, fluoxetine, fluvoxamine, nefazodone, paroxetine, sertraline HCl, and venlafaxine.

20. The method of claim 19 wherein the selective serotonin reuptake inhibitor (SSRI) sertraline HCl, fluoxetine, paroxetine or fluvoxamine.

21. The method of claim 19 wherein the selective serotonin reuptake inhibitor (SSRI) is sertraline HCl.

22. A method for the curative or prophylactic treatment of persistent selective serotonin reuptake inhibitor (SSRI) induced sexual dysfunction in an animal, including a human which comprises administering to the animal an effective amount of a phosphodiesterase type 5 (PDE5) inhibitor.

23. The method of claim 22 wherein the phosphodiesterase inhibitor is a 5-substituted pyrazolo [4,3-d]pyrimidine-7-one, a griseolic acid derivative, a 2-phenylpurinone derivative, a phenylpyridone derivative, a fused pyrimidine, a condensed pyrimidine derivative, a pyrimidopyrimidine derivative, a purine compound, a quinazolinone derivative, a phenylpyrimidone derivative, an imidazoquinoxalinone derivative or its aza analogue, a phenylpyrimidone derivative, a phenylpyridone derivative, a pyrimidopyrimidine derivative, a 4-aminoquinazoline derivative, a 4,5-dihydro-4-oxo-pyrrolo[1,2-a]quinoxaline derivative or its aza analogue, a polycyclic guanine derivative, a nitrogenous heterocyclic compound, a 2-benzyl-polycyclic guanine derivative, a quinazoline derivative, a 6-heterocyclyl pyrazolo [3,4-d]pyrimidin-4-one, a benzimidazole, a cycloheptimidazole, or an N-containing heterocycle.

24. The method of claim 23 wherein the 5-substituted pyrazolo [4,3-d]pyrimidine-7-one is 1-[[3-(6,7-dihydro-1-m ethyl-7-oxo-3-propyl-1H-pyrazolo[4,3-d]pyrimidin-5-yl)-4-ethoxyphenyl]sulfonyl]-4-methylpiperazine.

25. The use of a phosphodiesterase type 5 (PDE5) inhibitor for the manufacture of a medicament for the curative or prophylactic treatment of selective serotonin reuptake inhibitor (SSRI) sexual dysfunction in an animal, including a human.

26. The use of claim 25 wherein the phosphodiesterase inhibitor is a 5-substituted pyrazolo [4,3-d]pyrimidine-7-one, a griseolic acid derivative, a 2-phenylpurinone derivative, a phenylpyridone derivative, a fused pyrimidine derivative, a condensed pyrimidine derivative, a pyrimidopyrimidine derivative, a purine compound, a quinazolinone derivative, a phenylpyrimidone derivative, an imidazoquinoxalinone derivative or its aza analogue, a phenylpyrimidone derivative, a phenylpyridone derivative, a pyrimidopyrimidine derivative, a 4-aminoquinazoline derivative, a 4,5-dihydro-4-oxo-pyrrolo[1,2-a]quinoxaline derivative or its aza analogue, a polycyclic guanine derivative, a nitrogenous heterocyclic compound, a 2-benzyl-polycyclic guanine derivative, a quinazoline derivative, a 6-heterocyclyl pyrazolo [3,4-d]pyrimidin-4-one, a benzimidazole, a cycloheptimidazole, or an N-containing heterocycle.

27. The use of claim 25 wherein the phosphodiesterase inhibitor is a 5-substituted pyrazolo [4,3-d]pyrimidine-7-one or a 6-heterocyclyl pyrazolo [3,4-d]pyrimidin-4-one.

28. The use of claim 27 wherein the compound of formula (I): 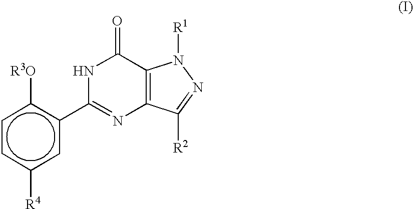
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆ -cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶ or CO₂R⁷; C₂-C₄ alkenyl optionally substituted with CN, CONR⁵R⁶ or CO₂R ⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²) piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy) C₂-C₆ alkyl; (R¹³R¹⁴N)C₂-C₆ alkyl; (R¹³R¹⁴NOC) C₁-C₆ alkyl; CONR¹³R¹⁴; CSNR¹³R¹⁴; or C(NH)NR¹³R¹⁴; and
R¹³ and R¹⁴ are each independently H; C₁-C₄ alkyl; (C₁-C₃ alkoxy)C₂-C₄ alkyl; or (hydroxy)C₂-C₄ alkyl; or a pharmaceutically acceptable salt thereof, or a pharmaceutical composition containing either entity.

29. A kit for the curative or prophylactic treatment of a serotonergic associated disorder in an animal, including a human, which comprises a selective serotonin reuptake inhibitor (SSRI) and a phosphodiesterase type 5 (PDE5) inhibitor.

30. The kit of claim 29 wherein the phosphodiesterase inhibitor is a 5-substituted pyrazolo [4,3-d]pyrimidine-7-one, a griseolic acid derivative, a 2-phenylpurinone derivative, a phenylpyridone derivative, a fused pyrimidine derivative, a condensed pyrimidine derivative, a pyrimidopyrimidine derivative, a purine compound, a quinazolinone derivative, a phenylpyrimidone derivative, an imidazoquinoxalinone derivative or its aza analogue, a phenylpyrimidone derivative, a phenylpyridone derivative, a pyrimidopyrimidine derivative, a 4-aminoquinazoline derivative, a 4,5-dihydro-4-oxo-pyrrolo[1,2-a]quinoxaline derivative or its aza analogue, a polycyclic guanine derivative, a nitrogenous heterocyclic compound, a 2-benzyl-polycyclic guanine derivative, a quinazoline derivative, a 6-heterocyclyl pyrazolo [3,4-d]pyrimidin-4-one, a benzimidazole, a cycloheptimidazole, or an N-containing heterocycle.

31. The kit of claim 30 wherein the phosphodiesterase inhibitor is a 5-substituted pyrazolo [4,3-d]pyrimidine-7-one or a 6-heterocyclyl pyrazolo [3,4-d]pyrimidin-4-one.

32. The kit of claim 31 wherein the phosphodiesterase inhibitor is a compound of formula (I): 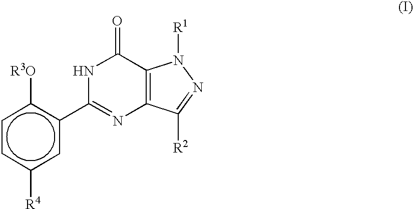
wherein R¹ is H; C₁-C₃ alkyl; C₁-C₃ perfluoroalkyl; or C₃-C₅ cycloalkyl;
R² is H; C₁-C₆ alkyl optionally substituted with C₃-C₆ cycloalkyl; C₁-C₃ perfluoroalkyl; or C₃-C₆ cycloalkyl;
R³ is C₁-C₆ alkyl optionally substituted with C₃-C₆-cycloalkyl; C₁-C₆ perfluoroalkyl; C₃-C₅ cycloalkyl; C₃-C₆ alkenyl; or C₃-C₆ alkynyl;
R⁴ is C₁-C₄ alkyl optionally substituted with OH, NR⁵R⁶, CN, CONR⁵R⁶, or CO₂R⁷; C₂-C₄ alkenyl optionally substituted with CN, CONR⁵R⁶ or CO₂R⁷; C₂-C₄ alkanoyl optionally substituted with NR⁵R⁶; (hydroxy)C₂-C₄ alkyl optionally substituted with NR⁵R⁶; (C₂-C₃ alkoxy)C₁-C₂ alkyl optionally substituted with OH or NR⁵R⁶; CONR⁵R⁶; CO₂R⁷; halo; NR⁵R⁶; NHSO₂NR⁵R⁶; NHSO₂R⁸; SO₂NR⁹R₁₀; or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form a pyrrolidinyl, piperidino, morpholino, 4-N(R¹¹)-piperazinyl or imidazolyl group wherein said group is optionally substituted with methyl or OH;
R⁷ is H or C₁-C₄ alkyl;
R⁸ is C₁-C₃ alkyl optionally substituted with NR⁵R⁶;
R⁹ and R¹⁰ together with the nitrogen atom to which they are attached form a pyrrolidinyl, piperidino, morpholino or 4-N(R¹²) piperazinyl group wherein said group is optionally substituted with C₁-C₄ alkyl, C₁-C₃ alkoxy, NR¹³R¹⁴ or CONR¹³R¹⁴;
R¹¹ is H; C₁-C₃ alkyl optionally substituted with phenyl; (hydroxy)C₂-C₃ alkyl; or C₁-C₄ alkanoyl;
R¹² is H; C₁-C₆ alkyl; (C₁-C₃ alkoxy)C₂-C₆ alkyl; (hydroxy) C₂-C₆ alkyl; (R¹³R¹⁴N)C₂-C₆ alkyl; (R¹³R¹⁴NOC) C₁-C₆ alkyl; CONR¹³R¹⁴; CSNR¹³R¹⁴; or C(NH)NR¹³R¹⁴; and
R¹³ and R¹⁴ are each independently H; C₁-C₄ alkyl; (C₁-C₃ alkoxy)C₂-C₄ alkyl; or (hydroxy)C₂-C₄ alkyl; or a pharmaceutically acceptable salt thereof, or a pharmaceutical composition containing either entity.

33. The kit of claim 29 wherein the selective serotonin reuptake inhibitor (SSRI) is citalopram, fluoxetine, fluvoxamine, nefazodone, paroxetine, sertraline, and venlafaxine.

34. The kit according to claim 33 wherein the selective serotonin reuptake inhibitor (SSRI) is sertraline, fluoxetine, paroxetine or fluvoxamine.

35. The kit according to claim 34 wherein the selective serotonin reuptake inhibitor (SSRI) is sertraline.

36. A composition for the curative or prophylactic treatment of a serotonergic associated disorder in an animal, including a human, which comprises a selective serotonin reuptake inhibitor (SSRI) and a phosphodiesterase type 5 (PDE5) inhibitor.

37. A method for the curative or prophylactic treatment of antidepressant induced sexual dysfunction in an animal, including a human which comprises administering to the animal an effective amount of a phosphodiesterase type 5 (PDE5) inhibitor.
